(19) United States
(12) Patent Application Publication
(10) Pub. No.: US 2017/0256581 A1
(43) Pub. Date: Sep. 7, 2017

(54) DETECTOR PACK AND X-RAY CT APPARATUS

(71) Applicant: Toshiba Medical Systems Corporation, Otawara-shi (JP)

(72) Inventors: Toshiya NAKAYAMA, Kawasaki (JP); Hideo NISHIUCHI, Yokohama (JP); Mayumi SO, Kamakura (JP); Satoru ASAGIRI, Yokohama (JP)

(73) Assignee: Toshiba Medical Systems Corporation, Otawara-shi (JP)

(21) Appl. No.: 15/446,498

(22) Filed: Mar. 1, 2017

(30) Foreign Application Priority Data
Mar. 2, 2016 (JP) 2016-040090

(51) Int. Cl. H01L 27/146; A61B 6/00; A61B 6/04; A61B 6/03

(52) U.S. Cl. CPC H01L 27/14661; H01L 27/14663; H01L 27/14636; H01L 27/14623; A61B 6/032; A61B 6/461; A61B 6/467; A61B 6/0407; A61B 6/4435

(57) ABSTRACT

According to an embodiment, a detector pack comprises a first substrate and a second substrate. the first substrate includes a first surface and a second surface. the first substrate is provided with an X-ray detecting element in the first surface. the second substrate includes a third surface and a fourth surface. The second substrate is disposed in the second surface to face the third surface. The second substrate is provided with a data acquisition circuit in the third surface. The first substrate and the second substrate are formed as a stacked body. The data acquisition circuit is provided in the third surface not to come in contact with the second surface of the first substrate.

### CROSS-REFERENCE TO RELATED APPLICATIONS

This application is based upon and claims the benefit of priority from the prior Japanese Patent Application No. 2016-040090, filed Mar. 2, 2016 the entire contents of which are incorporated herein by reference.

### FIELD

Embodiments described herein relate generally to a detector pack which detects an X-ray, and an X-ray CT apparatus which includes the detector pack.

### BACKGROUND

A medical X-ray CT (Computed Tomography) apparatus includes a base portion where a subject is placed, an annular gantry which is formed to contain the base on the inside, and a console which includes a monitor to display a state of the subject.

The gantry includes an X-ray tube which emits the X-ray toward the subject, and a detector unit which detects the X-ray transmitting the subject. The detector unit includes a plurality of detector packs which are disposed in an arc shape along a rotation direction of the gantry.

The detector pack includes, for example, a main substrate which is configured by a ceramic material, an optical semiconductor element which is disposed on the main substrate, a scintillator which is disposed in the optical semiconductor element, and a data acquisition system. The data acquisition system is, for example, an ASIC (Application Specific Integrated Circuit) which collects an electrical signal output by the optical semiconductor element, forms an electrical signal to be used in image processing on the basis of the collected electrical signal, and outputs the electrical signal to be used in the image processing. In addition, the detector pack is connected to a control substrate of an image processing device through a flexible printed circuit equipped with a connector. The image processing device includes the control substrate, and an ASIC which is mounted on the control substrate, and an image processing circuit.

The detector pack and an X-ray CT apparatus are necessarily increased in number of pixels in order to obtain an X-ray image in a wide range or with a high quality. An increase in number of pixels causes an increase of the electrical signals which are output by the optical semiconductor element. In a case where a plurality of data acquisition systems are provided to meet such a request, the data acquisition systems and the connectors occupy a large area on the substrate, and the mounting area may be insufficient. Therefore, a detector pack is desirably required to secure the mounting area of the components.

### BRIEF DESCRIPTION OF THE DRAWING(S)

FIG. 1 is a perspective view illustrating an X-ray CT apparatus according to a first embodiment;

FIG. 2 is a side view illustrating a detector pack of the X-ray CT apparatus which is partially drawn in cross-sectional view;

FIG. 3 is a top view of the X-ray detector pack;

FIG. 4 is a side view illustrating a detector pack according to a second embodiment which is partially drawn in cross-sectional view; and

FIG. 5 is a side view illustrating a detector pack according to another embodiment which is partially drawn in cross-sectional view.

### DETAILED DESCRIPTION

According to an embodiment, a detector pack comprises a first substrate and a second substrate. the first substrate includes a first surface and a second surface. the first substrate is provided with an X-ray detecting element in the first surface. the second substrate includes a third surface and a fourth surface. The second substrate is disposed in the second surface to face the third surface. The second substrate is provided with a data acquisition circuit in the third surface. The first substrate and the second substrate are formed as a stacked body. The data acquisition circuit is provided in the third surface not to come in contact with the second surface of the first substrate.

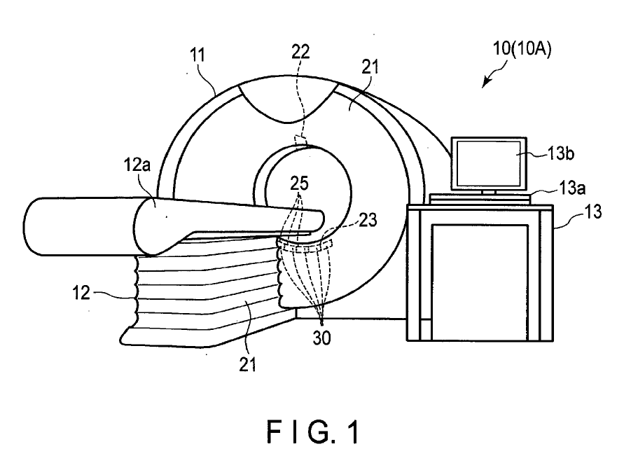

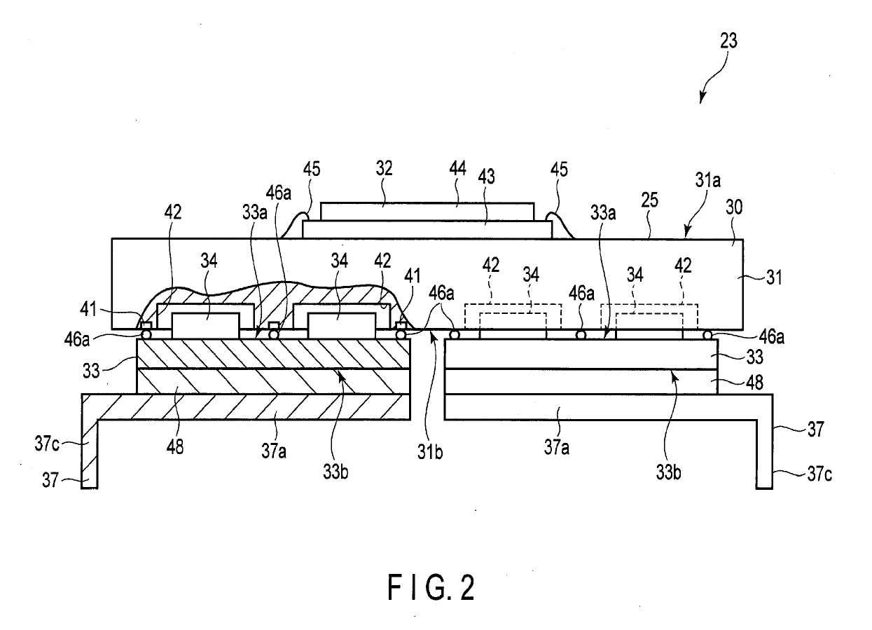

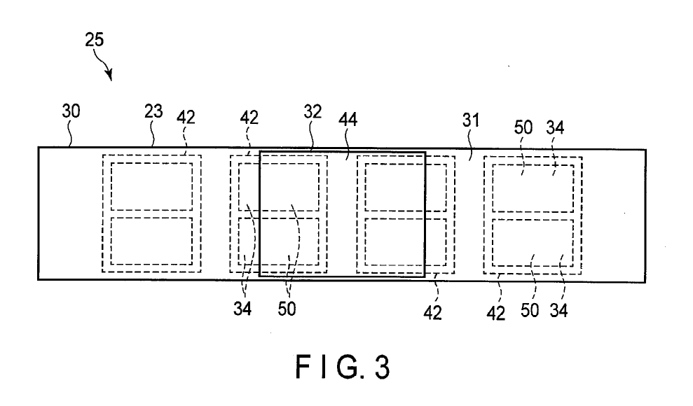

Hereinafter, an X-ray CT apparatus 10 according to a first embodiment will be described with reference to FIGS. 1 to 3. FIG. 1 is a perspective view illustrating the X-ray CT apparatus 10. FIG. 2 is a side view illustrating the detector pack which is partially drawn in cross-sectional view, and FIG. 3 is a top view of the detector pack. In each drawing, some parts may be enlarged or reduced in size, or may be omitted for the convenience of explanation.

As illustrated in FIG. 1, the X-ray CT apparatus 10 includes a gantry 11 which emits the X-ray to a subject and detects the X-ray transmitting the subject, a base 12 on which the subject is placed, and a console 13 which controls the base 12 and the gantry 11 or through which an operation is input and an image is displayed.

The gantry 11 includes a rotational unit 21 formed in an annular shape, the X-ray emission unit 22 which is configured to emit the X-ray, and an X-ray detector 23 which is configured to detect the X-ray emitted from the X-ray emission unit 22.

The rotational unit 21 is formed to be rotatable around its axis line. The rotational unit 21 includes an almost cylindrical rotation frame with a bore that forms a field of view. As shown in FIG. 1, the X-ray emission unit 22 and the X-ray detector 23 which are arranged to face each other via the bore are attached to the rotation frame. The rotation frame is a metal frame made of a metal such as aluminum into an annular shape. The X-ray emission unit 22 and the X-ray detector 23 may be, for example, fitted in concave portions formed in the rotation frame or fastened using fasteners such as a screw.

The X-ray emission unit 22 includes an X-ray tube, generates the X-ray according to a tube voltage supplied from a high-voltage generation apparatus, and emits the X-ray toward the subject.

The X-ray detector 23 is disposed at a position facing the X-ray emission unit 22 of the rotational unit 21. The X-ray detector 23 includes a plurality of detector units 25 which are provided in parallel in a rotation direction. Each detector unit 25 includes a detector pack 30, an image processing device (not illustrated) which includes a third substrate 35 disposed outside a second substrate 33 of the detector pack 30, and a fourth substrate 37 which includes a connector to connect the third substrate 35 and the second substrate 33. In this embodiment, for example, 32 to 48 detector packs 30 are provided in parallel in the rotation direction.

As illustrated in FIGS. 2 and 3, each detector pack 30 includes a first substrate 31 which includes a first surface 31a and a second surface 31b facing each other, an X-ray detecting element 32 which is disposed on the first surface 31a of the first substrate 31, a plurality of second substrates 33 which are disposed on a side near the second surface 31b of the first substrate 31, and a plurality of data acquisition systems 34 which are mounted on a third surface 33a (one main surface) of each second substrate 33.

The first substrate 31 is a ceramic substrate which is made of a ceramic material and includes the first surface 31a and the second surface 31b (a pair of main surfaces facing each other). Wirings 41 are formed on each of the first surface 31a and the second surface 31b. An optical semiconductor 43 is provided as the X-ray detector unit 32 on the first surface 31a of the first substrate 31. In addition, a concave portion 42 is formed in the second surface 31b of the first substrate 31 to contain a part of the data acquisition system 34.

The concave portion 42 is dented along the shape of the data acquisition system 34 for example. In this embodiment, the concave portion 42 is, for example, a rectangular shape in cross-sectional view and has a certain depth as illustrated in FIG. 3. In this embodiment, four concave portions 42 are disposed in parallel on the second surface 31b, and two data acquisition systems 34 are disposed in each concave portion 42.

The optical semiconductor 43 includes a plurality of optical semiconductor elements which are disposed in a matrix shape as detector elements to detect the X-ray. Each optical semiconductor element is configured to convert light into an electrical signal, and to output the electrical signal. The optical semiconductor 43 is electrically connected to the wiring 41 of the first substrate 31 by wire bonding using wires 45. Electrical connection between the optical semiconductor 43 and the first substrate 31 is not limited to this. For example, the optical semiconductor 43 may be connected to the wiring 41 of the substrate 31 by a through-silicon via and a bump electrode. A scintillator 44 is mounted on the optical semiconductor 43.

The scintillator 44 is disposed in a matrix shape to face the optical semiconductor element. The scintillator 44 is configured to convert light into the X-ray, and to output the X-ray.

The second substrate 33 is an interposer 47 which is formed in a rectangular plate shape. An example of a material may include ceramics, silicon, and resin. In consideration of a temperature change at the time of driving, the ceramics is preferably because it has the same material as that of the first substrate 31 and has no thermal expansion difference.

The second substrate 33 includes the third surface 33a and a fourth surface 33b (a pair of main surface facing each other). The second substrate 33 is disposed such that the third surface 33a faces the second surface 31b of the first substrate 31. In this embodiment, two second substrates 33 are disposed to face the second surface 31b of the first substrate 31. Each second substrate 33 is smaller than the first substrate 31 and has a size enough to dispose four data acquisition systems 34. The second substrate 33 is electrically connected to the wiring 41 on the second surface 31b of the first substrate 31 using a bump 46a.

The third surface 33a of the second substrate 33 is mounted with a plurality of data acquisition systems 34 where a part thereof is contained in the concave portion 42. In addition, a first connector 48 of the fourth substrate 37 is connected on the fourth surface 33b of the second substrate 33.

The data acquisition system 34 is, for example, an ASIC 50. The ASIC 50 has a function of collecting the electric signal output by the optical semiconductor element, forming a signal to be used in the image processing on the basis of the electrical signal, and outputting the signal to be used in the image processing. For example, in this embodiment, the ASICs 50 are disposed in four columns in a first direction (a longitudinal direction of the first substrate 31) parallel to a rotation shape, and in two column in a second direction along the rotation direction (that is, total eight ASICs 50).

The data acquisition system 34 is, for example, mounted on the third surface 33a of the second substrate 33 through the bump 46a, and electrically connected to the wiring 41 of the second substrate 33. A part on a side of the data acquisition system 34 is contained in the concave portion 42 which is formed in the second surface 31b of the first substrate 31.

The first connector 48 and a second connector (not illustrated) are provided on both sides of the fourth substrate 37. For example, the fourth substrate 37 is a wiring substrate with flexibility. The fourth substrate 37 integrally includes a first connection portion 37a which is connected to the fourth surface 33b of the interposer 47 through the first connector 48, a second connection portion (not illustrated) which is connected to the main surface of a control substrate (not illustrated) through the second connector (not illustrated), and a continuous portion 37c between the first connection portion 37a and the second connection portion.

The first connector 48 is mounted on the fourth surface 33b of the second substrate 33, and electrically connected to the wiring 41 on the fourth surface 33b of the second substrate 33. In other words, the first connector 48 is electrically and mechanically connected to the second substrate 33. In other words, a ninth surface (one main surface in the first connection portion 37a of the fourth substrate 37) is connected to the fourth surface 33b of the second substrate 33 through the first connector 48. Even in a tenth surface (the other main surface of the first connection portion 37a of the fourth substrate 37), there is formed a mounting area where various types of electronic components such as an electronic circuit is mounted.

The second connector is mounted on the main surface of the third substrate 35, and electrically connected to the wiring on the third substrate 35. In other words, the second connector is electrically and mechanically to the third substrate 35.

In other words, in the detector pack 30, the first substrate 31, the data acquisition system 34, the second substrate 33, the first connector 48, and the fourth substrate 37 are disposed to be stacked in a predetermined stacking direction, and a mounting area is formed in the fourth substrate 37 to mount the components.

The image processing device includes a control substrate (the third substrate 35) which is mounted with various types of electronic components for image processing and includes an image processing circuit.

The image processing device is formed to receive a signal output by the ASIC 50, to perform the image processing on the basis of the received signal, and to output data obtained by the image processing. The base 12 includes a table top 12a which is movable and where a subject is placeable. The table top 12a is moved in a state where the subject is placed so that the subject is moved to pass through an emitting region of the X-ray.

The console 13 includes an input circuitry 13a and a display 13b. The input circuitry 13a includes a mouse, a keyboard, and a touch panel, and is configured to receive an input of operation information when an operator operates the base 12 and the gantry 11. The console 13 is a housing in which a control circuit which controls the operation of the base 12 and the operation of the gantry 11, a data transmission circuit, and a memory circuit are built on the basis of the operation information input to the input circuitry 13a.

The display 13b is, for example, a display which can display an image on the basis of the data output by the image processing device. The image displayed on the basis of the data output by the image processing device is an image of the inner portion of the subject which is formed on the basis of the X-ray transmitting the subject.

Next, the operation of the X-ray CT apparatus 10 will be described. When the subject is placed on the table top 12a of the base 12, the operator operates the input circuitry 13a to start the operation of the X-ray CT apparatus 10

When the operation of the X-ray CT apparatus 10 starts, the table top 12a of the base 12 moves in a predetermined direction in a space inside the gantry 11, and the X-ray emission unit 22 emits the X-ray.

Each detector pack 30 of the X-ray detector 23 detects the X-ray transmitting the subject. Specifically, the X-ray transmitting the subject enters each scintillator 44. Each scintillator 44 receives the X-ray to generate the light. Each optical semiconductor element of the optical semiconductor 43 converts the light generated by the scintillator 44 into the electrical signal. The optical semiconductor element of the optical semiconductor 43 outputs the electrical signal. The electrical signal output from the optical semiconductor element is transmitted to the ASIC 50 through the wiring 41 of the first substrate 31 and the second substrate 33. The ASIC 50 collects the electrical signal output by the optical semiconductor element, and forms a signal suitable to the image processing on the basis of the electrical signal. In addition, the ASIC 50 outputs the formed signal. The signal output from the ASIC 50 is transmitted to the control substrate through the wiring 41 of the second substrate 33 and the fourth substrate 37.

The image processing device includes an image processing processor, the data transmission circuit, and the memory circuit, and performs the image processing on the basis of the signal formed by the ASIC 50. The image processing device transmits the data after the image processing to the console 13.

The console 13 receives the data formed by the image processing device. The display 13b displays an image of the inner portion of the subject which is formed on the basis of the received data.

The detector unit 25 and the X-ray CT apparatus 10 configured as above includes the second substrate 33 which faces the first substrate 31. The data acquisition system 34 and the connector 48 are mounted in the second substrate 33. Therefore, it is possible to secure an area for mounting the electronic components in the surface of the first substrate 31. In other words, the ASICs 50 are arranged in one main surface of the second substrate 33, and the connector 48 of the fourth substrate 37 is mounted in the other main surface. Therefore, the plurality of ASICs 50 can be mounted without increasing the first substrate 31.

In addition, in the detector unit 25 and the X-ray CT apparatus 10, the fourth substrate 37 is mounted in the main surface of the second substrate 33 through the first connector 48. Therefore, the components can be mounted onto the fourth substrate 37, so that the mounting area can be secured.

In addition, when the interposer 47 is used as the second substrate 33, the ASIC 50 can be disposed near the first substrate. Therefore, a distance from the optical semiconductor element to the ASIC 50 becomes short, and a distance of the electrical path where the electrical signal output by the optical semiconductor element passes up to the ASIC 50 can be shortened, so that it is possible to prevent noises.

In addition, the concave portion 42 is formed in the second surface 31b, and at least a part of the ASIC 50 is contained in the concave portion 42. Therefore, a DAS system 60 configured by the interposer 47 and the ASIC 50 can be disposed near the optical semiconductor element. Therefore, it is possible to shorten a distance from the optical semiconductor element to the ASIC 50 which is disposed in the interposer 47.

### Second Embodiment

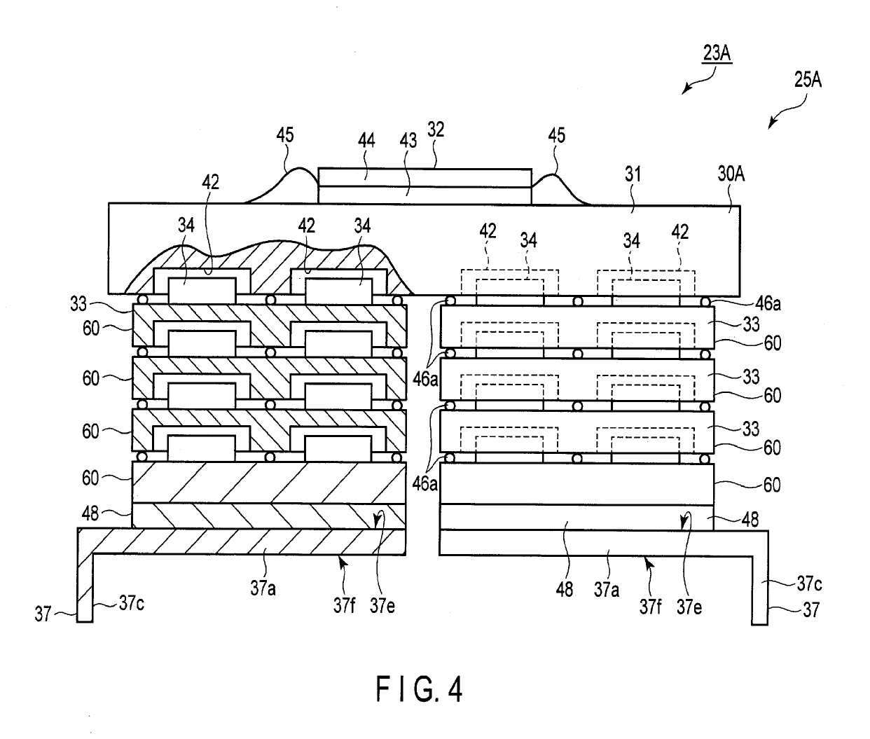

Hereinafter, an X-ray CT apparatus 10A according to a second embodiment will be described with reference to FIG. 4. FIG. 4 is a side view illustrating a part of a detector pack 30A of the X-ray CT apparatus 10A according to the second embodiment, in which a part of the drawing is illustrated in cross section view.

Further, the detector pack 30A according to the second embodiment is the same configuration as that of the X-ray CT apparatus 10 according to the first embodiment except that the second substrates 33 in which the ASIC 50 is mounted are provided in plural stages. Therefore, the same configurations of a detector unit 25A according to the second embodiment, the detector pack 30A, and the X-ray CT apparatus 10A as those of the first embodiment will be denoted by the same symbols, and the redundant description will be omitted.

The X-ray CT apparatus 10A according to this embodiment includes the gantry 11, the base 12, and the. console 13 which are configured similarly to those of the X-ray CT apparatus 10.

The gantry 11 includes a rotational unit 21 formed in an annular shape, the X-ray emission unit 22 which is configured to emit the X-ray, and an X-ray detector 23 which is configured to detect the X-ray emitted from the X-ray emission unit 22.

The rotational unit 21 is formed to be rotatable around its axis line.

An X-ray detector 23A is disposed at a position facing the X-ray emission unit 22 of the rotational unit 21. The X-ray detector 23A includes a plurality of detector units 25A which are disposed in parallel in the rotation direction. Each detector unit 25A includes the detector pack 30A, the image processing device which includes the third substrate 35 disposed outside the second substrate 33 of the detector pack 30A, and the fourth substrate 37 which includes a connector to connect the second substrate 33 in the lowest stage and the third substrate 35. In this embodiment, for example, 32 to 48 detector packs 30A are provided in parallel in the rotation direction.

As illustrated in FIG. 4, each detector pack 30A includes the first substrate 31 which includes the first surface 31a and the second surface 31b facing each other, the X-ray detector unit 32 which is disposed on the first surface 31a of the ceramic substrate, a plurality of second substrates 33 which are disposed to be stacked in plural stages on a side near the second surface 31b of the first substrate 31, and a plurality of ASICs 50 as the data acquisition system 34 which is mounted on the third surface 33a of each second substrate 33.

In other words, in this embodiment, the DAS systems 60, each of which is configured by the second substrate 33 and the ASIC 50 on the second substrate 33, are provided by stacking in plural stages. Among the second substrates 33 in plural stages, the first connector 48 of the fourth substrate 37 is mounted in the fourth surface 33b of the second substrate 33 farthest away from the first substrate 31 in the stacking direction. The second substrate 33 is connected to the control substrate of the image processing device through the fourth substrate 37.

The concave portion 42 in which a part of the ASIC 50 is disposed in the fourth surface 33b is formed in the second substrate 33 except the second substrate 33 connected to the fourth substrate 37. The second substrate 33 is mounted through the bump 46a. Therefore, the wiring 41 formed in the fourth surface 33b of the second substrate 33 is electrically and mechanically connected to the third surface 33a of the other second substrate 33 disposed to face the fourth surface 33b of the second substrate 33.

The X-ray CT apparatus 10A and the detector unit 25A according to this embodiment can also achieve the same effect as that of the first embodiment. In other words, the second substrate 33 mounted with the ASIC 50 is provided, and the connector is mounted in the second substrate 33, so that the mounting area of the first substrate 31 can be secured.

Further, in this embodiment, the second substrates 33 are stacked in plural stages to dispose the plurality of ASICs 50 in each stage, so that a lot of ASICs 50 can be provided without increasing the size of the device in a surface direction.

Further, in this embodiment, the optical semiconductor 43 and the scintillator 44 have been described for example as the X-ray detector unit 32 and the X-ray detecting element which convert and output the X-ray into the electrical signal, but the invention is not limited thereto. For example, in place of the optical semiconductor 43 and the scintillator 44, the X-ray detecting element which can directly convert and output the X-ray into the electrical signal may be used as the X-ray detector unit 32 and the X-ray detecting element.

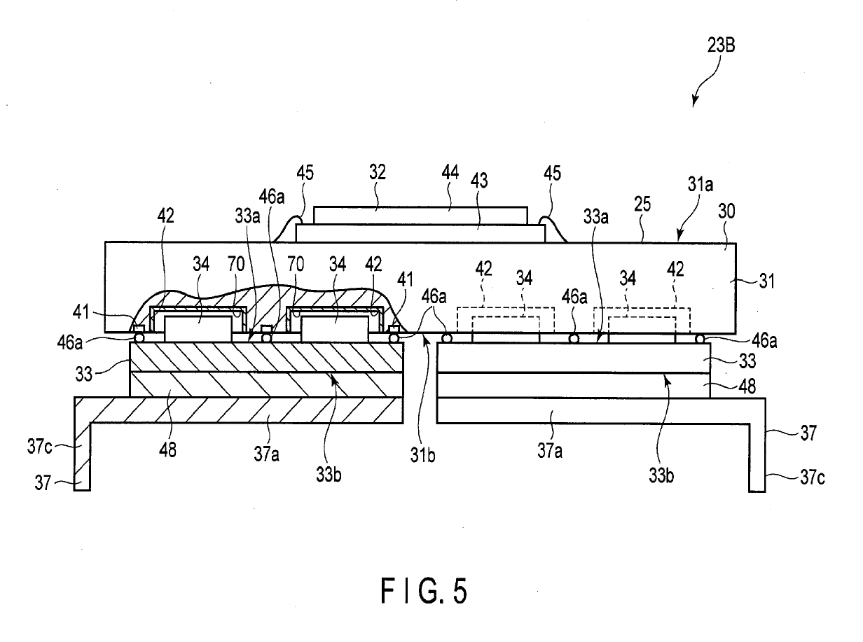

In addition, the X-ray detector may be configured to include a shielding plate 70 between the second surface 31b of the first substrate 31 and the ASIC 50 (data acquisition circuit). FIG. 5 is a side view illustrating an X-ray detector 23B according to another embodiment which is partially drawn in cross-sectional view. As illustrated in FIG. 5, the X-ray detector 23B has the same configuration as that of the X-ray detector 23 except that the shielding plate 70 is further provided between the second surface 31b of the first substrate 31 and the ASIC 50 (data acquisition circuit). The shielding plate 70 is a layer having a predetermined thickness, and configured by metal (for example, zinc, tungsten, molybdenum, and the like). The shielding plate 70 is provided in the inner wall of the concave portion 42 where the. ASIC 50 is contained. The shielding plate 70 shields the X-ray which is incident to the ASIC 50. According to this embodiment, the shielding plate 70 is provided in the concave portion 42, so that the ASIC 50 can be protected from a radiant ray, and a radiation resistance of the ASIC 50 can be increased.

The above described “processing circuitry” means, for example, a central processing unit (CPU), a graphics processing unit (GPU), an application specific integrated circuit (ASIC), a programmable logical device (e.g., a simple programmable logic device (SPLD), a complex programmable logic device (CPLD), and a field programmable gate array (FPGA)), or the like.

Note that programs may be directly incorporated in processing circuitry instead that programs are stored in storage memory. In this case, the processing circuitry reads programs incorporated in circuitry and executes the programs to realizes predetermined functions.

Each function (each component) in the present embodiment is not necessary to be corresponded to a single processing circuit and may be realized by a plurality of processing circuits. To the contrary, for example, at least two functions (at least two components) may be realized by a single processing circuit. Further, a plurality of functions (a plurality of components) may be realized by a single processing circuit.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A detector pack, comprising:
a first substrate that includes a first surface and a second surface, and is provided with an X-ray detecting element in the first surface; and
a second substrate that includes a third surface and a fourth surface, is disposed in the second surface to face the third surface, and is provided with a data acquisition circuit in the third surface,
wherein the first substrate and the second substrate, are formed as a stacked body, and
the data acquisition circuit is provided in the third surface not to come in contact with the second surface of the first substrate.

2. The detector pack according to claim 1, further comprising: a fourth substrate that transmits data processed by the data acquisition circuit to a third substrate,
wherein the first substrate ,the second substrate ,and the fourth substrate are formed as the stacked body, and
the second substrate and the fourth substrate are electrically connected.

3. The detector pack according to claim 2, the fourth substrate is formed of a flexible printed circuit.

4. The detector pack according to claim 2, further comprising:
a third substrate that is connected to the second substrate by the fourth substrate; and an image processing circuit that is provided in the third substrate.

5. The detector pack according to claim 1,
wherein the X-ray detecting element includes an optical semiconductor element that is disposed on the first surface, electrically connected to the first substrate, and converts and outputs light into an electrical signal, and a scintillator that is provided in the optical semiconductor element, and converts and outputs an X-ray into light, and
wherein the data acquisition circuit is an ASIC that is electrically connected to the second substrate, collects the electrical signal output by the optical semiconductor element, and outputs an electrical signal used in image processing.

6. The detector pack according to claim 2,
wherein the X-ray detecting element includes an optical semiconductor element that is disposed on the first surface, electrically connected to the first substrate, and converts and outputs light into an electrical signal, and a scintillator that is provided in the optical semiconductor element, and converts and outputs an X-ray into light, and
wherein the data acquisition circuit is an ASIC that is electrically connected to the second substrate, collects the electrical signal output by the optical semiconductor element, and outputs an electrical signal used in image processing.

7. The detector pack according to claim 1,
wherein a concave portion is formed in the second surface of the first substrate, and
wherein at least a part of the data acquisition circuit is contained in the concave portion.

8. The detector pack according to claim 1, further comprising:
data acquisition units stacked in plural stages, each of which includes the second substrate and the data acquisition circuit in a side near the second surface of the first substrate, and
a connector disposed on the fourth surface of the second substrate of at least any one of the plurality of data acquisition unit,
wherein an electronic component is configured to be mounted in a surface on an opposite side of the connector of the flexible printed circuit.

9. The detector pack according to claim 4, further comprising:
data acquisition unit, each of which includes the second substrate and the data acquisition circuit stacked in plural stages in a side near the second surface of the first substrate, and
a connector disposed on the fourth surface of the second substrate of at least any one of the plurality of data acquisition unit,
wherein an electronic component is configured to be mounted in a surface on an opposite side of the connector of the fourth substrate.

10. The detector pack according to claim 5, further comprising:
data acquisition unit, each of which includes the second substrate and the data acquisition circuit stacked in plural stages in a side near the second surface of the first substrate, and
a connector disposed on the fourth surface of the second substrate of at least any one of the plurality of data acquisition unit,
wherein an electronic component is configured to be mounted in a surface on an opposite side of the connector of the fourth substrate.

11. The detector pack according to claim 7, further comprising:
data acquisition unit, each of which includes the second substrate and the data acquisition circuit stacked in plural stages in a side near the second surface of the first substrate, and
a connector disposed on the fourth surface of the second substrate of at least any one of the plurality of data acquisition unit,
wherein an electronic component is configured to be mounted in a surface on an opposite side of the connector of the fourth substrate.

12. The detector pack according to claim 8,
wherein a concave portion is formed in a fourth main surface of at least any one of the plurality of data acquisition unit, and at least a part of the data acquisition circuit is contained in the concave portion.

13. The detector pack according to claim 2,
wherein an electronic component is mount in a surface on an opposite side of the surface facing the fourth surface of the fourth substrate.

14. The detector pack according to claim 4,
wherein an electronic component is mount in a surface on an opposite side of the surface facing the fourth surface of the fourth substrate.

15. The detector pack according to claim 5,
wherein an electronic component is mount in a surface on an opposite side of the surface facing the fourth surface of the fourth substrate.

16. The detector pack according to claim 7,
wherein an electronic component is mount in a surface on an opposite side of the surface facing the fourth surface of the fourth substrate.

17. The detector pack according to claim 12,
wherein an electronic component is mount in a surface on an opposite side of the surface facing the fourth surface of the fourth substrate.

18. The detector pack according to claim 1, further comprising:
a shielding plate that is provided between the second surface of the first substrate and a shielding plate which is provided between the data acquisition circuits and blocks an X-ray.

19. An X-ray CT apparatus, comprising:
an X-ray detector provided with a plurality of detector pack according to claim 1; and
an X-ray tube that emits an X-ray.
